(19) United States
(12) Patent Application Publication
(10) Pub. No.: US 2008/0113368 A1
(43) Pub. Date: May 15, 2008

(54) METHOD OF MODELING DNA MOLECULES

(76) Inventor: Harry MONTAGUE, Brookline, MA (US)

Correspondence Address: KENYON & KENYON LLP, RIVERPARK TOWERS, SUITE 600, 333 W. SAN CARLOS ST., SAN JOSE, CA, 95110, US

(21) Appl. No.: 11/965,138

(22) Filed: Dec. 27, 2007

(51) Int. Cl. C12Q 1/68; G06F 19/00

(52) U.S. Cl. 435/6; 702/20

(57) ABSTRACT

A set of relationships using properties of the DNA molecule and its hydrogen bridge electron cycloid motion tied to the Fibonacci-Lucas series concepts and a complex number Argand diagram to provide a computer-implemented set of numbers to describe the number of DNA molecular bases traveling away from a starting point, the number of DNA molecular divisions away from the starting point molecule, and the triplet letter selection occurring at the new location.

### BACKGROUND OF THE INVENTION

The appearance of the Fibonacci series and the “golden number” in nature suggests that relationships coming from this mathematics may be connected to the DNA molecule. The regularity of the DNA strand suggests a very formal way of dealing with the constantly changing bases. The genetic code has always suggested a very strong logical system to reduce the 64 possible letter combinations down to 20 amino acids.

### BRIEF DESCRIPTION OF THE DRAWINGS

FIG. 1 is a top scaled view of the DNA helix projected onto one plane, with the C-G bases showing a theoretical single straight line representing the helix perimeter.

FIG. 1a is the same as above but with the T-A bases.

FIG. 2 is a scaled diagram of combined Fibonacci and Lucas spirals into one spiral shown with their separate boxes used to form them.

FIG. 3 is a table of numbers showing the correct pairing of the Fibonacci and Lucas series and how they form new identical series.

FIG. 4 is a scaled diagram of the Fibonacci and Lucas spirals with the same origin, pulled apart in a special manner so they can be divided.

FIG. 5 is a plan view of the start of a Theodorus √1 spiral.

FIG. 6 is the expanded plan view of this spiral showing only the ends of the √1 triangles that form the spiral and these triangles organized into three sub-spirals.

FIG. 7 shows half a cycloid curve formed with points on a rolling circle with a ground line divided into four equal segments.

FIG. 8 shows this half cycloid curve as it relates to half of a perfect sine wave curve.

FIG. 9 shows the equal length tie lines between the half cycloid and the half perfect sine curve.

FIG. 10 shows the projection of these equal length ties onto two half circles.

FIGS. 11 through 20 show the genetic code and amino acid functions related to complex conjugate numbers.

FIG. 11 shows an example of the method by which the 64 complex conjugate numbers are plotted on the Argand diagram.

FIG. 12 shows the full 64 complex conjugate numbers plotted on the Argand diagram.

FIGS. 12a and 12b show the extension of the comparative lines of FIG. 12.

FIG. 13 shows the conjugate values labeled on the upper set of plotted points.

FIG. 14 shows the actual real values of the plotted points labeled on the points and tied to the sloping line.

FIG. 15 is a table showing the 64 conjugate points tied to their comparative numbers; their amino acids; and their group functions.

FIGS. 16a-16c are a series of tables tying the amino acid-conjugate numbers to their letter combinations.

FIGS. 17a-17b show the amino acid and letter combinations transferred to the format of FIG. 13.

FIGS. 18a-18b show the same set of amino acid and letter combinations with the above format related to their ammo acid group functions.

FIG. 19 shows the hydrophobicity values as they relate to FIGS. 13-18b amino acids.

FIG. 20 shows the Protein occurrence values as they elate to the amino acids.

### DETAILED DESCRIPTION

The Fibonacci-Lucas series (the Fibonacci series and the Lucas series springing from a common relationship, but a different starting point) offers a mathematical reason why the DNA molecule can constantly divide and reproduce itself over many generations. The counterbalancing of the two series against each other, providing opposite-directional control, perfectly suits the way the DNA molecule works. The flat geometry of the helix projections tied to the hydrogen bridge offers an explanation as to why the DNA molecule can operate with separate ladder levels joined only at the outer perimeter. This geometry is also tied to the Theodorus √1 spiral recording in a manner to bring every new ladder step back to the origin. These two flat geometries lead us to the genetic code on the two dimensional geometry of the Argand plane and its possible multi-dimensional interpretation. The series of DNA ladder steps must have a means of using the RNA messenger and transcription molecules. This means must expand the possible combinations record for the amino acids within the Protein molecule. The complex numbers provide this means and with their unique last letter organization explain many of the functions of the amino acids.

The DNA molecule takes the geometric form of a very long double helix with parallel ladder rungs connecting the two sides of the helix at a joint in the middle of the rung that can be easily divided. As viewed from the top, these parallel ladder rungs come into the strands or outer chains and are skewed in very slightly varying angles depending on which bases are used to connect to the side strands. There are four possible bases, labeled A, T, G, and C, that are used, and they vary at each ladder rung. Their order can be in any of an enormous number of different possibilities.

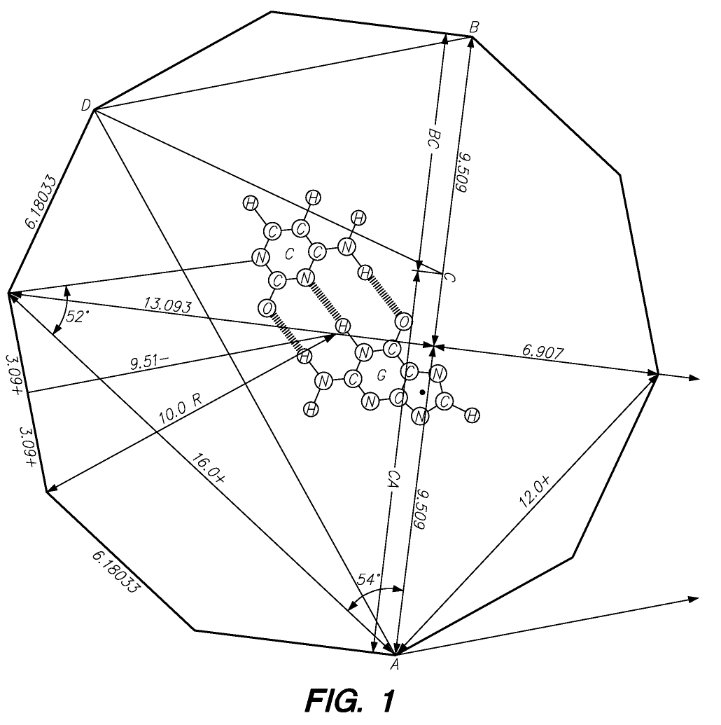

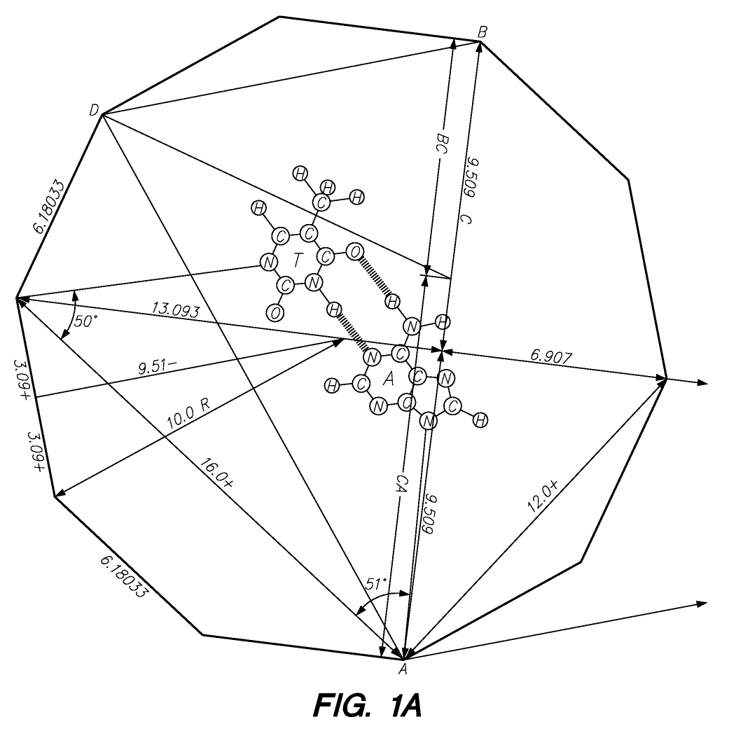

FIG. 1 shows the condition where bases C, connected to G, are joined at the side chain. FIG. 1 a shows the condition where bases T, connected to A, are joined at the side chain. Not drawn is the condition where an average of the joining angles of the bases is used; this average option would have a very similar joining angle to the C base. In dealing with the outer chain, it is known that a DNA molecule takes 10 base revolutions to complete one turn of the helix circle as shown in FIG. 1. A theoretical single straight line is shown to represent the center of the chain molecules joining the base points. The B-DNA helix molecule, one of a number of variations, generally has a diameter of approximately 20.0 nm, and thus has a radius of approximately 10.0 nm.

In FIG. 1, if all 10 pie sections had been drawn, each would form a 360/10=36 degree angle at the center; when this angle is bisected it forms two equal pie sections each with angles of 18 degrees, 72 degrees, and 90 degrees. In these right triangles, each one would have the sine of 18 degrees which is 0.309, thus the triangles would have sides of 9.51 nm, 10.0 nm and 3.09 nm. The whole pie section would have an outer chord of 3.09×2=6.18 nm. Let us suppose the angle is still 18 degrees and the sine 3.09, but the diameter is 20.00064 nm and not 20.0 nm. The triangle using the radius of 10.00032 nm has two other sides of 9.50998 nm and 3.0901699 nm. The whole pie section would now have an outer chord of 3.0901699×2=6.180339887 nm.

As many people know 0.6180339887--, one tenth of the chord size, is the “golden number,” also called phi, which may be derived from the Fibonacci series, where, as n becomes a very large integer, Fib(n−1)/Fib(n) converges to phi. If an outer point on the DNA chain represents a base ladder connection and is given a Fibonacci number, then to go up to the next base connection above this, the starting number would be multiplied by 6.180339887/10, or phi. Thus moving up the DNA chain, as represented on the horizontal plane of both FIGS. 1 and 1a, involves moving along one 6.180339887 chord, 10 chords completing the circumference of the outer chain. FIGS. 1 and 1a are horizontal recording systems that are tracking the vertical helix chain spinning away from it. Each step on the DNA ladder has its own FIG. 1 or 1a, which could also have starting G or T base connecting angles, but not, as the drawings show, a different size of chord or of the projected flat diameter of the helix. The Fibonacci series is the mathematics that records the various ladder step positions but it does not in any way imply or predict whether the bases are C, G, T, or A.

FIGS. 1 and 1a show a horizontal recording system for each step of the DNA ladder. The DNA ladder as is known, records in groups of 3 bases or triplets. FIGS. 1 and 1a show the first and fourth bases directly connected with a line that works out to be approximately 16.0+nm. If the outer circle of the helix had been drawn, and if this fourth point is connected to the sixth point, the far end of the diameter line, a right triangle within a semi-circle would be formed. This right triangle would have 3, 4, and 5 proportion sides, whether we use a diameter of 20.0 nm or 20.00064 nm. If the diameter length used is 20.0 nm, the triplet chord line is 16.00 nm, and the short chord 12.0 nm. If this triangle is bisected with line a dropping down perpendicular to the diameter line, this line will be 9.509645 nm long as determined by triangulating it. This triangulation is done using the radius of the helix and the opposite segment on the bisected diameter.

When this bisecting line is extended to the far side, the total line shown as B-A on FIGS. 1 and 1a is 2×9.509645×2=19.01929 nm. On FIGS. 1 and 1a, chord point D is shown and if the angle BDA is bisected and the bisecting line drawn, it hits line BA at point C. Then the ratio of BC to AC would be the “golden ratio”, 1.6180339887 which is 1 over the golden number 0.6180339887. Add 1 to 0.6180339887 for the reciprocal, 1.6180339887, and add 1 to this number for 2.180339887 which is its square. So adding 1 to the ratio of BC to AC can form its square. Here is an example where lines can be squared.

Going back to the bisecting 9.509645 nm line which hits the diameter—this line divides the diameter into two parts, as calculated before, one 13.093 nm long, the other 6.907 nm for a total of 20.0 nm, ignoring the 0.00064 nm correction. 13.093/9.509645=9.509645/6.907 and is known as a “mean proportional” leading to 9.509645×9.509645=6.907×13.093 or 9.5096452=the divided diameter portions of 6.907×13.093. So we have a means of squaring numbers as related to a bisected 3, 4, 5 right triangle within a semi-circle. If the semi-circle shrinks or expands, even 0.000064 nm or much larger sizes for different types or sizes of the DNA molecule, these relationships still hold true. Finally, this also means 9.509645=(6.907×13.093). The lengths 13.093, 6.907, and 9.509645 are said to be in a harmonic progression because if these lines were strings and taut and could vibrate, in theory the frequencies of this vibration would differ by equal intervals. All the ladder rungs are separated by equal intervals or distances up the DNA helix, thus reinforcing the concept of equal intervals.

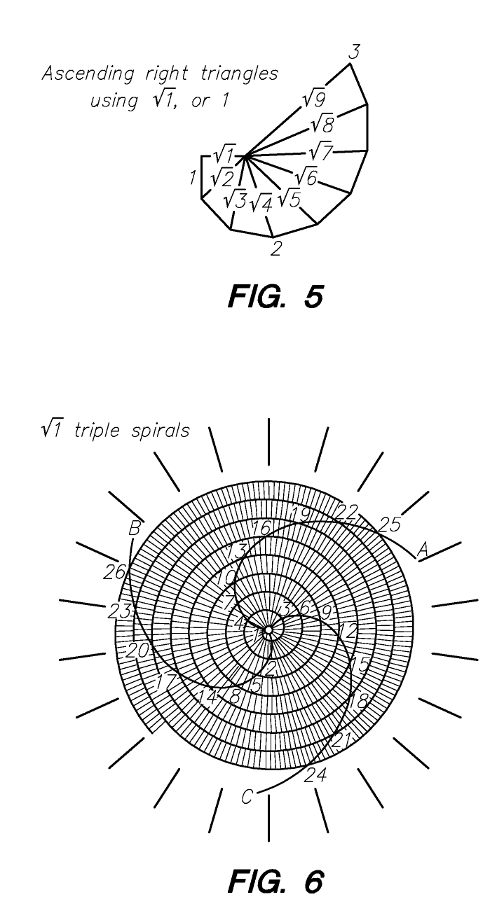

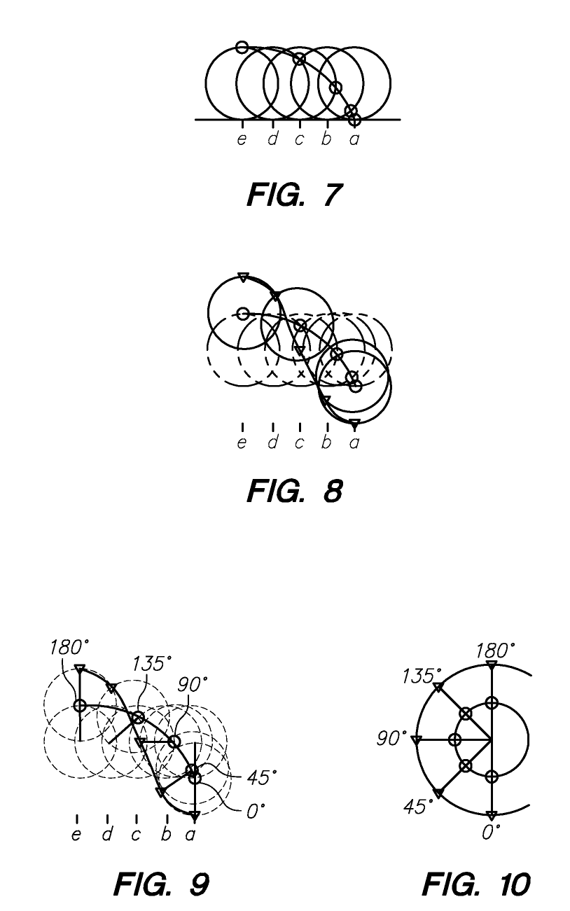

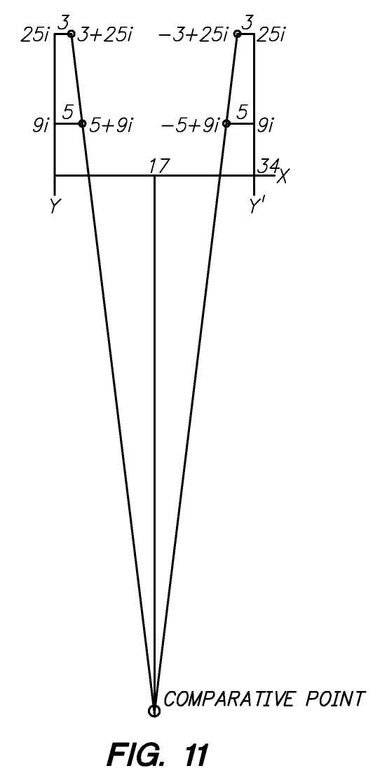

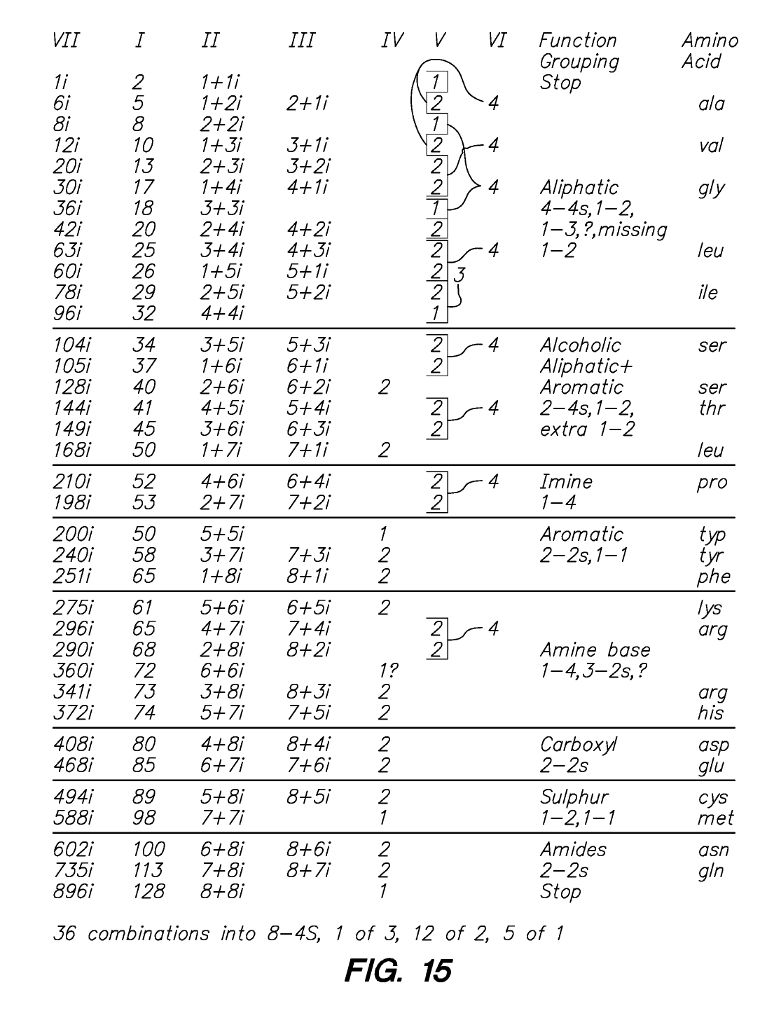

The equal intervals derived from the harmonic progression are further reinforced by later work, dealing with equally spaced intervals of square root numbers as shown in FIG. 6. FIG. 6 is a diagram of a Theodorus spiral which gives us a means for ascribing consecutive integers taken from the sea of non-square integers, each integer representing a step on the DNA ladder. FIGS. 7-10 deal with equal intervals of frequencies of waves derived from uniform cycloids taken from the hydrogen bridge. The final genetic code work goes back to square roots in FIGS. 11 through 21 in this case dealing with negative square roots, and imaginary and complex numbers. Overall, in order to tie the flat geometry of FIG. 1 to the above-mentioned concepts, it is very important to have found the “mean proportional”, that forms squares and square roots.

As stated before, each base, at each ladder rung, is connected to the outer chain at a slightly different angle. Since the outer chain does not vary and has no way of anticipating what the next rung base will be, it would suggest that the ladder structure, in order to accommodate these different connection angles, would be designed for an average rung condition. Specifically between the centerline of the individual base and the triplet tying line, there is an angle formed at the point on the chord perimeter as shown on FIG. 1. If the conditions of the changing base angles were shown, the angles would be: T=50 degrees, sine=0.766, A=51 degrees, sine=0.777, C=52 degrees, sine=0.788, G=54+degrees, sine=0.810. The total of the sine values equals 3.141 or very close to the value of π and since there are four bases, the average equals 3.141592653/4 or π/4. The average angle of the bases between their centerlines and the triplet chain chord lines when translated into a sine function, equals π/4=0.7854=52 degrees, which is a little smaller than 0.788, the sine of 52 degrees on the C base.

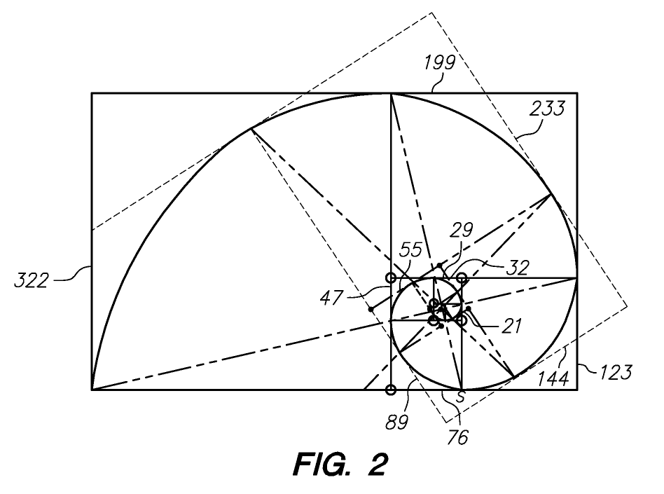

The Fibonacci spiral is shown in FIG. 2, with its growing series of squares in dash lines which control the size increasing spiral curve. (Shown also, overlapping the Fibonacci spiral, is the identical Lucas spiral but it is tied to a different set of squares as shown in solid lines.) Looking at each separate Fibonacci square with two of its sides next to and housing the arc of the spiral curve, we see that these arcs within the squares are also, in fact, ¼ of the circumference of a circle. The arcs then belong to two geometries, the geometry of a spiral and that of a set of circles growing in size. The whole circle circumference would be π×d, so a quarter of it would be (π×d)/4 and if d=1, then this arc segment would be π/4. Again looking at FIG. 2, each arc segment has a distinct curve but when they are put together they form a continuous spiral. The separate pieces of arc are like each distinct step of the DNA ladder which is part of the continuous helix chain. It should be recalled that each arc segment is a part of both a Fibonacci and a Lucas continuous spiral because these spirals overlap each other. Added to this, because both the average base chain connection and the Fibonacci-Lucas arcs relate to π/4 ratios, there is a very strong tie between the Fibonacci-Lucas numbers and the chain structure of the DNA molecule.

The golden number 0.6180339887 which, as mentioned before, has become well used in science over time, may be derived from a large Fibonacci number divided into the number just ahead of it in the series, as noted earlier. This ratio comes closer and closer to phi as the numbers become larger. The golden number comes from (√5−1)/2 which is an endless number. The Fibonacci series: 1, 1, 2, 3, 5, 8, 13, 21, 34, etc. is related to the Lucas series 1, 3, 4, 7, 11, 18, 29, 47, etc. in that both series come from the relationship G(n+1)=G(n)+G(n−1), where G=Fib(n) or G=Lucas(n). In the Fibonacci sequence, Fib(0)=1, Fib(1)=1, Fib(2)=2, and so on. In the Lucas sequence, Lucas(0)=2, Lucas(1)=1, Lucas(2)=3, and so on. (The ratio of successive numbers in the Lucas series also converges to the golden number as the numbers become larger.) This product of the two series also become more accurate as the numbers grow. The two series should be lined up correctly as shown below:

| | | | |
|---|---|---|---|
| | 3 | 7 | |
| Adding (going | 5 | 11 | subtracting (going |
| down a column) | | | up a column) |
| | 8 | 18 | |

Use any three consecutive Fibonacci numbers tied correctly to their consecutive Lucas numbers and the sum of alternate Fibonacci numbers equals the difference of alternate Lucas numbers; 3+8=18−7, 5+13=29−11, etc. More generally, Fib(n−1)+Fib(n+1)=Lucas(n+1)−Lucas(n−1), or Fib(n−1)+Fib(n+1)=Lucas(n). If 3, 5, 8, the Fibonacci numbers are positions on one chain and 7, 11, 18 Lucas numbers are positions on the opposite chain, then adding down the 3, 5, 8, side and back up the 7, 11, 18 side would form a loop. The DNA molecule is known to use loops to relate its two opposite sides. This is another example of how the two series can be used to keep track on the base step positions on the DNA ladder. Lined up properly, first we find the product of the two series: 1×1=1, 1×3=3, 2×4=8, 3×7=21 etc. Then we add the products together, then add 1, 1+3+8+21=33+1=34, and we get a Fibonacci number. This simple multiplication plus the addition of the product plus 1 can be done with any consecutive set of the series numbers, anywhere in the series, to produce a cross-over from one series to the other. Instead of adding 1, if 2 is added there is an even simpler cross-over from one series to the other. 3×3×5=45+2=47, 8×8×5=320+2=322 etc. If we start with our DNA chain chord number divided by 10, 0.6180339887 and add 1, we get 1.6180339887, or Phi, the golden ratio, which is the reciprocal of 0.6180339887, or phi, the golden number. If we add 2, we get 2.6180339887 which is the square of 1.6180339887. So we can perform the reciprocal and squaring functions by crossing over from one side of the base to the other and adding either 1 or 2. Multiplying the series numbers together and producing a useful product suggests there may be a larger picture that can be obtained by this multiplication process.

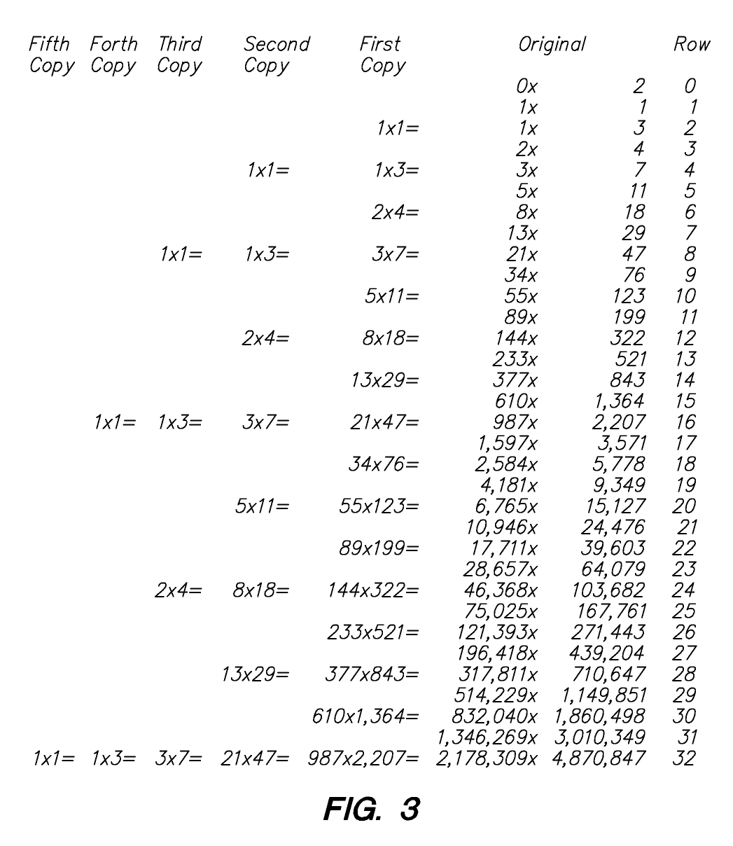

FIG. 3, which is a chart, shows the two series put together in the correct way and the product of the two series as they produce another series of Fibonacci numbers. This multiplication can be continued and produce not just one new set of combined series but many copies of the combined series, the only limitation being the lack of infinite area on the pieces of paper for the new numbers. Each new copy is identical to the original series which is why they are called copies, but their numbers in the columns are spaced farther apart than in the original column.

Because of this different spacing, the identical combined sets obviously do not line up in rows with their matching sets on the original or other copies. (Notice the spacing between combined sets doubles each time.) Again, it should be understood that any Fibonacci-Lucas number represents a position on a certain row. Number, 2,178,309--4,870,847 are on row 32 of the original set, the 32nd DNA base position away from the starting 0 row. Each new copy is marked First Copy, Second Copy, Third Copy, etc. when one moves to the left of the original set. On bottom row 32 on the chart, there are six generations of numbers: 1×1,---1×3,---3×7,--21×47,-987×2207,--2,178,309×4,870,847. But it should be remembered that even as the numbers get huge, they only represent their position, their DNA base step position counting from zero. As was stated at the outset, if you multiply a Fibonacci number by √5 you get a Lucas number--thus 3×3×5=45, adding 2 for the Lucas number equals 47. Now this time add 2 again and 47+2=49; then for the second part of the process return back to the starting line by taking the square root of 49 for 7, the Lucas number that goes with 3. With the small numbers, remember the multiplying process using √5 was not accurate, but in this case we have found a way to exactly cross between Fibonacci and Lucas numbers.

In FIG. 1 we derived the “mean proportional” segments and the square root of numbers tying it again to the two series of numbers. In the above example of going up the Fibonacci numbers and crossing over to the Lucas numbers and taking the square root to come back, is extremely important. The numbers in the two series are aligned in an opposite directions, in the same manner as the 3′ and 5′ strand and the 5′ and 3′ strands of the DNA molecule. It also permits the two series like the two strands to separate with leading and lagging positions. This allows a division and pairing up in the particular manner which the Fibonacci-Lucas series numbers can thus match.

If the Fibonacci-Lucas numbers of FIG. 3 are divided down the middle, as the DNA molecule does, the Fibonacci series would become spread out in order to take a new matching series of Lucas numbers. The combined Fibonacci-Lucas spiral of the FIG. 2 numbers must also pull apart in a logical way so the two parts can form new partners. On the solid lines forming the Lucas boxes shown in FIG. 2, on the right bottom end of the box with 322 sides is a tiny circle. Again on the left leg of box 199 is another tiny circle, and again on the upper leg of box 123, again on 76, to 47, down to 29; the very small numbers not having the tiny circles. These tiny circles are the radius centers for the arcs of the Lucas spiral. Place a sheet of tracing paper over FIG. 2 and copy only the Lucas boxes and their radius points and the spiral that goes with those radius points. Remember, on FIG. 2 the spiral line is the same for both the Fibonacci and Lucas series but imagine that these identical superimposed spiral lines can be separated.

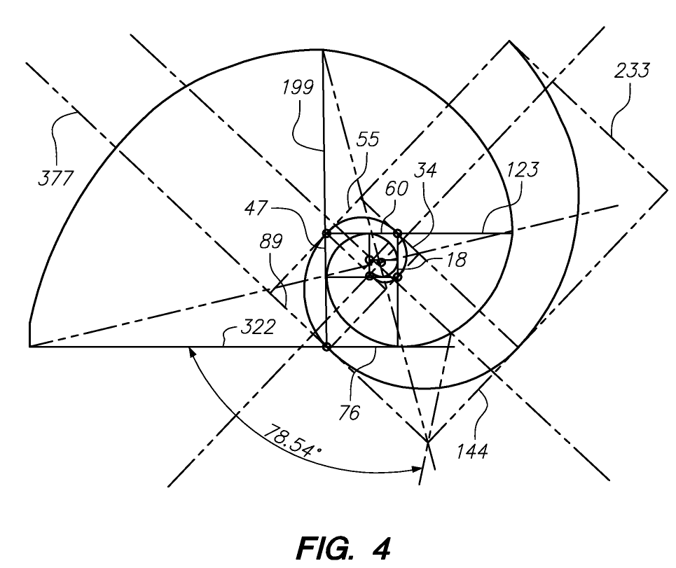

Place the pencil point on the origin point of the entire spiral and rotate the tracing paper counterclockwise 78.5398 degrees. Now look at the dotted box lines of the Fibonacci squares under the tracing paper. Notice that the radius center of the Lucas square 322 has now fallen on the outer circumference point of the Fibonacci square 144. Next the Lucas radius center point 199 is on the outer Fibonacci circumference point 89, etc. And so on down. FIG. 4 is showing the result of this rotation of the tracing paper and the new relationships formed by the juxtaposition of the two drawings put together. Now the cross connection between the Fibonacci and Lucas series at one point will allow their two box line segments to come apart at that point. Because of the rotation, when this happens notice that the entire Lucas box 322 now does not overlap the entire Fibonacci box 144, allowing the separation to occur. Each joined Fibonacci-Lucas series number like F322-L144, F199-L123 represents a step on the DNA ladder. Then rotating the two spirals so they can be separated occurs because the center point of the Fibonacci spiral falls on the leg intersection point of the Lucas spiral. This 78.5398 degree rotation is of course, π/(4×10) again showing the tie between the circumference and the radius lines generating them with 4, π, and 10.

FIG. 2 is a flat spiral, a two dimensional expanded geometry tied to the flat helix projection of FIG. 1. FIG. 5 and its expanded form, FIG. 6, are again flat spirals which also can be shown to tie to FIG. 1. FIGS. 5 and 6, Theodorus spirals of triangles as mentioned earlier, are tied to the square root construction of right triangles within the semi-circles of FIG. 1. In these figures, the √1 set of growing triangles produces the basic slow growing spirals from which whole integers can be pulled out as shown in FIG. 6. These whole integers fall on 22 radial lines which are indicated only in part on the perimeter of the drawing. These integers on the radial lines are spaced every 22 whole integer units, there being 7 slow growing spiral arcs between the 22 unit jumps in integer values--7×3.14285714----equals 22, or 22/7=3.14285714----. In FIG. 6, the three steep spirals listing the integers spaced three units apart would relate to the triplets of the DNA molecule. Like each row in FIG. 2, each integer of FIG. 6 represents a step on the DNA ladder.

What is also important to realize is that FIG. 5 growing to FIG. 6 is one of four possible Theodorus spirals that could exist. Look at FIG. 5, the first right triangle 1, √1, and √2 and imagine the √1 line extending straight through the origin point and forming a −√1, line which would be part of an invert triangle −√1, −1, and −√−2. Thus this triangle would be the starting triangle of a−1 spiral which would have whole negative integers forming steep spirals spaced in between the positive step spirals of FIG. 6. The other two Theodorus spirals would be formed from √−1 and √−1 triangles. These four spirals √1, −√1, √−1, and −√−1 would make it possible to relate the whole integers within these spirals to the four quadrants of complex numbers. When we look at FIG. 6 we forget that the partial lines between the slow spiral arcs represent the sides of triangles that extend all the way back to the origin. The most critical property of square root triangle spirals is that no matter how large the integers get, they are tied with their triangle side line back to the point of origin. Think of any set of large numbers and realize that the Theodorus spiral can incorporate them and relate them back to zero geometrically. The Theodorus spirals form the most geometric counting systems one can think of.

The three step spirals of FIG. 6 being tied to 7 and 22 or 11×2, these numbers can be algebraically tied to the Fibonacci-Lucas series as shown below:

| Fibonacci series | Lucas series | Fibonacci series | Lucas series |
|---|---|---|---|
| 5 × 7 = 35 − 1 = 34 | 11 × 7 = 77 − 1 = 76 | 8 × 11 = 88 + 1 = 89 | 18 × 11 = 198 + 1 = 199 |
| 8 × 7 = 56 − 1 = 55 | 18 × 7 = 126 − 3 = 123 | 13 × 11 = 143 + 1 = 144 | 29 × 11 = 319 + 3 = 322 |
| 13 × 7 = 91 − 2 = 89 | 29 × 7 = 203 − 4 = 199 | 21 × 11 = 231 + 2 = 332 | 47 × 11 = 517 + 4 = 521 |
| 21 × 7 = 147 − 3 = 144 | 47 × 7 = 329 − 7 = 322 | 34 × 11 = 374 + 3 = 377 | 76 × 11 = 836 + 7 = 843 |
| 34 × 7 = 238 − 5 = 233 | 76 × 7 = 532 − 11 = 521 | 55 × 11 = 605 + 5 = 610 | 123 × 11 = 1353 + 11 = 1364 |

The above group of numbers is just one example of how the Fibonacci and Lucas series are intertwined with 7 and 11.

The square roots of FIGS. 5 and 6, like the golden number, can be tied to a reciprocal ratio. If the irrational square roots above and below a whole integer square are subtracted, the difference equals one over the whole integer.

√224=14.96629547

√225=15 15.03329637−14.96629547=0.06666683= 1/15=0.06666683

√226=15.03329637

The average or uniform value π/4 as stated earlier relates to the joint of the base and side chain, the arc of the Fibonacci-Lucas spiral, and the geometry of separating these spirals. It can be found in yet another location on the DNA molecule, namely at the weak bond in the middle of the ladder rungs. As shown before, in the complete ladder structure, each rung has two sides made of the two bases joined in the middle that are easily divided. Also, for molecular bonding reasons the A and T bases are always joined together as are the G and C bases. The weak bond at the middle between the bases has a hydrogen atom joined to one base which has an electron that jumps back and forth between this atom and the opposite base. If that bond were a strong bond, that electron would jump across and back in a straight line, but because the bond is weak, it is known that the electron behaves as if the path it is moving on is a cycloid path and not a straight path. If you put a point touching the ground on a circle with a diameter of 1 unit, that point, as the circle rolls, will draw a cycloid path of a length of 4 units over a ground distance of πunits. The circumference of that circle=π×d. When d=1, the circumference, π, equals the ground line length. So the electron behaves as if it is traveling 4 units even though it is only π units away from the opposite side. In general terms, the electron behaves as if it is traveling over the ratio of 4/π units, but if the actual distance is more than 1 unit, the cycloid increases proportionally to 4/π. 4/π, of course is just the inverse of all the above mentioned π/4 functions, and the mathematics to create inverse relationships comes with the use of the golden number. Even more basic, is the concept that when the hydrogen bridge's weak 4/π bond is working with the base side chain π/4 angle, the product formed by this cooperation will relate to 4/π to π/4 and thus 4/π×π/4=1.

When the A and T bases at each rung level are joined, there are two hydrogen bonds side by side in the flat plane of the ladder rungs. When the G and C bases are joined, there are three side by side bonds, these three having two different sizes from each other and from the bond distance between the A and T bases. The chances of something randomly crossing all these bonds would be the average of all the different distances. This actual average distance can be worked out using the average overall hydrogen bridge sizes minus the fixed covalent bond size. This covalent bond is the distance between the hydrogen bridge and the joining atom. Thus 0.103 is a strong bond and always remains constant. From the textbook numbers: the two A-T base hydrogen bridge lengths 0.28+0.30=0.58/2=0.29 nm average, the three G-C hydrogen bridge lengths 0.29+0.30+0.29=0.88/3=0.29333 nm average, together 0.29+0.29333=0.58333/2=0.291665−0.103, the covalent bond=0.188665 nm. I felt it was possible that on the G-C hydrogen bridges the top two would be more frequently used, so this average could be slightly increased to 0.1887574. (This increase would be proportional to increasing the diameter from 20.0 nm to 20.01 nm.) If this change could be accepted then the average electron would be crossing a distance of 0.1887574×4/pi=0.240333+the covalent bond 0.103=0.34333 for the total hydrogen bridge size. If the total distance is divided in 10 equal spaces 0.34333/10=0.034333, then 3 would be the fixed hydrogen bond 3×0.034333=0.103 and 7 would be the average cycloid size, 7×0.034333=0.240333. If these sizes are written out in long form: 0.03433333333/0.2403333333=0.142857142857−namely, 1/7.

If we go back to our earlier calculations, the large unbisected right triangle within the semi-circle 20.0 nm long diameter hypotenuse has its right angle at point A. Using the semi-circle, when a tangent line is drawn through point A, shown partially on FIG. 1, and is extended, it would intersect the extension of the 20.0 nm diameter. Calculating this tangent line, it would be 30.0 nm long, 1½ times as long as the diameter. It too would have rooted relationships to the geometry within the limit lines of the chain. This tangent line may describe one of a number of possible ties of the DNA molecule to the RNA messenger molecule pushing up against it. Finally all the numbers are tied to a right-hand B-DNA with its 20.0 nm diameter. If there are variations with the B-DNA, or the A-DNA and Z-DNA with possible helix diameters of 23.7 nm, 25.5 nm, and 18.4 nm, the ratio of the principal members, chord 6.180339887 nm, with 20.0 nm diameter would remain constant but the size of the members would shrink or expand as needed to exist in these variations. Since all these ratios and the geometry of FIG. 1 exist in a flat plane, changes in the vertical rise between bases would not affect them.

One of the near misses in all this mathematics is included here as a curiosity item:

4/7×π/4=1=(20.01×0.24033333)/(0.343333×3×4.669)

20.01/3=6.67−6.18=0.49×10²=7²

Mitchell Feigenbaum's delta number, known to relate to living processes, is 4.6692016091--however, and not 4.669, and the diameter 20.01 nm doesn't work with the chord size of 6.180339887 nm which is the golden number and not 6.18 nm. However, if we rework the bottom equation with exact numbers we come closer than expected. Delta, 4.6692016091, divided by 0.7 equals 6.67028813, and if the golden number 6.180339887 is subtracted from this, it equals 0.489948126 which when subtracted from 0.49 equals 0.000051874. (0.49 is 0.7².) With the above equations, may be close enough to tie the delta number to the golden number.

In FIG. 7, half a cycloid motion is shown with a circle having a point and rolling on a ground line. The ground line is arbitrarily divided into four segments tied to the four sections on the cycloid line. At the points where the segment lines cross the rolling circle circumferences, small circles have been shown. In FIG. 8 these same circular points have been used as the center point of a new set of circles on which are marked small triangles which divide the formed sine curve into four segments. FIG. 9 shows the small circular points connected to the small triangle points with a radius length line. If there had been an infinite number of tiny segments on the ground line tied to the infinite number of segments on the cycloid, connected with the infinite number of radius lines, a sine curve with an infinite number of points would have been formed. In other words the perfect, unique, sine wave is created from the cycloid.

FIG. 10 is the projection of the original four radius lines with their small circular and triangle points drawn onto two half circles. What this shows is that the radius lines marked 0 degrees, 45 degrees, 90 degrees, 135 degrees, and 180 degrees are turning at a uniform rate, even though the cycloid segment size and the sine wave segment size are not uniform. We started with a uniform division of a base line and we ended up with a uniform division of the circumference of two circles, one half as big as the other. This is a unique geometric means for transferring the number of divisions from a straight line onto an equal number of divisions on the circumference of a circle. The size of the divisions from the straight line to the circle circumference will always be a ratio of an integer to π.

The hydrogen bridge in the form of the cycloid path ratio together with and times, the average chain sine angle was equal to unity, 4/π×π/4=1. But the electron is known to exist only on the one side by going over and coming back every time. This would be a total traveled distance of (2×4)/7 or 8/7 going over and back on the 7×0.034333=0.240333 nm average electron gap. The integers 7 and 8 are linked with squares, and inverse ratios and possibly other constants. 8/7=1.14285714 . . . =10/7, which is straightforward. In less straightforward mathematics: (82×2+72×11)/10²=6.67.

However, the delta number 4.6692016091 divided by 0.7 equals 6.67028813 and if the golden number 6.180339887 is subtracted from this, it equals 0.489948126 which when subtracted from 0.49 equals 0.000051874. In other words the relationship between the delta number and the “golden number” misses by this very tiny margin. The projected side chain length, 6.1803398874/3.4 the distance between ladder steps=1.88177 or 1.82 units out for each unit up which produces a slope of approximately 29 degrees.

In FIG. 10, the four divisions of the large semi-circle when compared to the π/2 distance of the half ground line would form again the ratio of π/4. However, this time if the chosen base line were divided into 10 equal spaces, then the circumference of the two circles would be divided into 10 equal arcs. Then if the base line of the total hydrogen bridge was divided into 10 equal 0.034333 segments, 3 would be for the covalent bond, 3×0.034333=0.103, and 7 for the average cycloid size, 7×0.034333=0.240333. The ratio of 3 units to 7 units on the hydrogen bridge has meaning but taking 1 or more units within the 7 units has no meaning because the electron on the cycloid path exists only at either end of the path. Having said this, if this ratio could be related to the 10 equal divisions of the DNA helix circumference as shown on FIG. 1, then the covalent bond length would relate to the 3 base unit, matching the triplet construction of the DNA molecule. Both the triplet and the covalent bond are fixed quantities and not subject to change.

Consider the DNA helix circumference as a circle and not a series of chords as shown in FIGS. 1 and 1a. The length of the circumference would be 20.0 nm×π. This circumference is also divided into 10 equal segments by the 10 chords. Each one of these segments has a length of 20π/10=2π. Place a small circle with unit diameter on one of these segments. If an epicycloid, a circle rolling on a curved surface and producing a cycloid shape, of the size mentioned were to roll back and forth, its cycloid length would relate back to the diameter of the whole helix. The diameter is tied to the hydrogen bridge and the other relationships within the geometry of the helix. This small circle could be a means of taking all this information to the outer circumference of the DNA molecule and the RNA molecule pressing against it.

FIGS. 7 to 10 represent the introduction of a novel geometry tying straight lines to circle circumference, but for this paper more significantly, they represent the introduction of wave action to the hydrogen bridge process. The reason the cycloid behavior of the electron gap is known is because of the wave patterns it gives off. Now we know what that wave pattern looks like. Tying this new perfect wave to the hydrogen bridge, if the actual straight distance of the electron gap is used, and not the average 0.1887574 nm, then the cycloid size working with the 4/πratio would change. Changing the cycloid size causes the wave size to change which is tied to the cycloid in an unchanging manner. Thus we have the perfect uniform wave size and the changing wave sizes based on the different electron gap sizes. Until now we have dealt with average uniform conditions related to the structure of the DNA molecule. Dealing with changing base angles and the electron gaps that go with them and comparing this change to the uniform structure and the uniform wave guiding system is a holographic concept. Working with this concept we have finally started dealing with elements that form the code of the DNA molecule.

Back to considering the question of different electron gap sizes, at the center hydrogen bonds, when the A and T bases are used, it does not matter which side the A and the T bases are on. They can switch sides and the size of the electron gap does not change. But if and when this information in the form of a wave were to travel to the side chain it would have to pass through different joining angles depending on whether it was an A or T connection. Information joining an A base at a chain sine angle of 0.777 can go across a hydrogen bridge length of either 0.28 or 0.30; information joining a T base at a chain angle of 0.766 can also go across the same lengths. Information joining a C base at a chain sine angle of 0.788 can go across a hydrogen bridge length of either 0.29 or 0.30, and information joining a G base chain sine angle of 0.8105 can go across the same lengths. This gives us eight possible alternatives that can be selected at each step of the ladder for the transfer of information. (The C-G bases have three bridges but two of their sets have the same bridge value, 0.29, so they were not listed as different alternatives even though all three were used in the average bridge size calculations.)

The DNA molecule works in triplets of bases but the first two of these bases are fundamentally key in the selection of the amino acids used; the third base is somewhat redundant in comparison to the first two bases. If the first base has eight possible alternatives, then the second base also has another eight possible alternatives. Taken together, assuming the third base is completely redundant, the triplet formed has 64 possible combinations for passing information up the chain and out to the messenger RNA molecule. There are 64 letter combinations that produce the genetic code, so the above suggestion of using the different hydrogen bridge alternatives to tie to the selection of amino acids is an interesting possibility. Obviously, the third base factors into the possible alternative replacing of the first and second bases in some manner. The known genetic code has its own irregularities and degeneracy so this last suggestion is highly possible. We have tied two useful pieces of information coming from two different areas of the molecule. At each base then, working with actual side chain angles and the actual hydrogen bridge values we can place a combined mathematical value for each of the eight possible alternatives mentioned above. Going up the DNA ladder, 7/8=0.875, the sine of 29 degrees, the slope of the DNA helix.

The geometric presentation of the complex numbers used to derive the genetic code can be seen on the Argand diagrams of FIGS. 11, 12, 12a, 12b, 13, and 14. The complete list of the 64 possible combinations of complex numbers that are grouped together to form 20 possibilities are shown on Table 15, in columns II and III. On these columns, with real and imaginary numbers using integers from 1 to 8, there are 28 sets of numbers in column II and 28 sets of numbers in column III where the real and imaginary integers have been switched and there are 8 with identical real and imaginary integers. Shown in column II, these complex numbers are in fact complex conjugate numbers which means that, as an example, in column II, 3+5i shown is combined with its conjugate 3-5i multiplied together to form (3+5i)×(3−5i)=9−(25×−1)=9+25=34. In the same line but in column III, 5+3i is (5+3i)(5−3i)=25+9=34. The two sets of identical complex conjugate products are shown in column I.

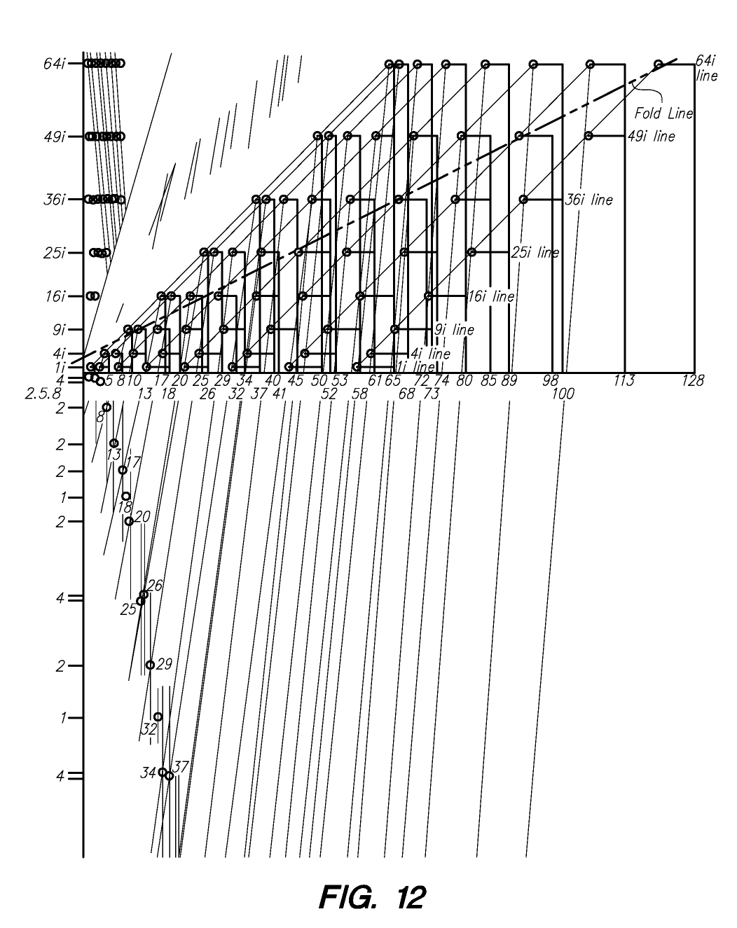

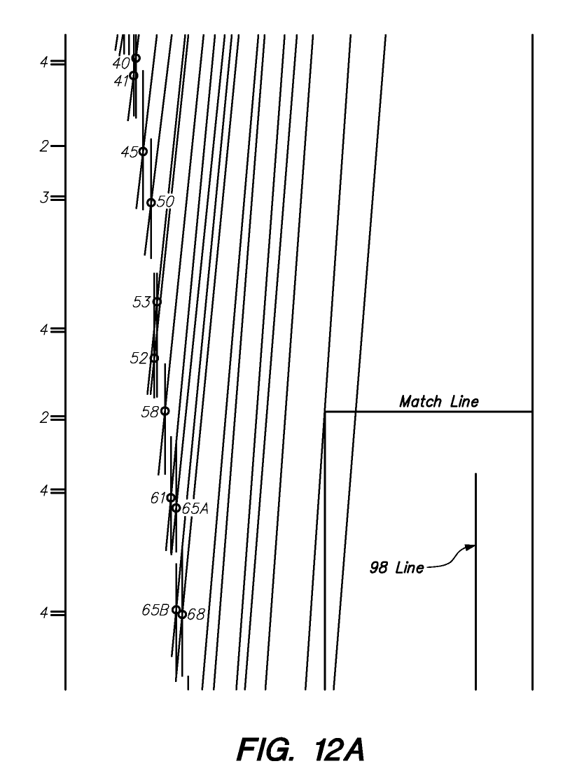

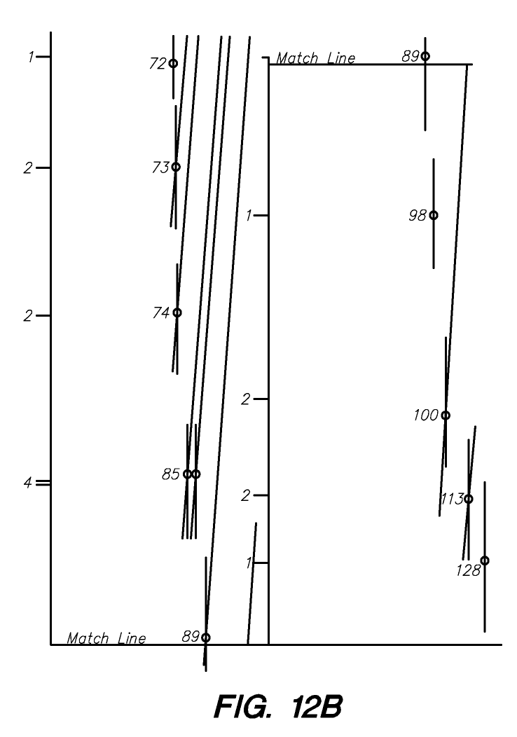

Using the above example of complex numbers, FIG. 11 shows the imaginary integers of 3+5i. When 5 is squared it becomes 25i, and the complex number 3+25i is plotted at the top of the left hand imaginary y axis and the base real x axis. Similarly in the accompanying complex number 5+3i, the integer 3 is also squared to become 9i, and the complex number 5+9i is also plotted below the first complex number. These two plotted numbers are joined with a line which is extended downwards. Continuing on FIG. 11, measure out along the x axis the real number distance using conjugate 34 as mentioned before in Table 15, column I. Perpendicular to this line form another y axis called y′. Off this axis to the left in the negative direction plot, −3+25i and −5+9i in the same manner as you plotted 3+5i and 5+9i. Again, join them with a line that extends downward. This line and the line coming from 3+25i and 5+9i will meet at a point of the figure called the comparative point. If a perpendicular line extending down from the midpoint on the x axis between the two sets of numbers at 34/2=17 is drawn, it too will pass through the comparative point. FIG. 11 shows the method by which all the complex numbers of Table 15 are plotted on FIG. 12. Look at the set of complex numbers plotted off the y axis through the conjugate point 34, −3+25i and −5+9i. First, this set of paired conjugate numbers uses the square of the imaginary integer but not the real integer. In fact working with the y axis and moving the real number integers to this axis, squares the real integer. Second, the top numbers actually coordinate. Measuring in real numbers using the x axis as a guide, −3 is added to 34 or 31, lining up on the 25i y axis, thus having a coordinate position of 31+25i. In a similar manner, the bottom number would be −5 added to 34 or 29, lining up on the 9i y axis, and thus having a coordinate position of 29+9i.

As we said earlier, there are 8 sets of complex numbers that have the same real and imaginary integers, 1+1i, 2+2i, 3+3i, etc. If these numbers are plotted in the same manner as 3+25i and 5+9i, squaring the their imaginary integer, then calculating their coordinate positions as we did above, the list of the plotted coordinate points would be: 1+1i, 6+4i, 15+9i, 28+16i, 45+25i, 66+36i, 91+49i, and 120+64i. These coordinate complex number points are plotted on FIG. 12 and connected with a dashed line. The coordinate complex numbers 2, 8, and 18 curve somewhat downward ending at 2 and deviate from the straight line that can be drawn through the rest of the numbers. The slope of this remaining group is 1.82 units out along the x axis for each 1 unit up on the y axis. Using this slope as a guide, the coordinate points are: 32 is 28/16i=1.75, 50 is 45/25i=1.8, 72 is 66/36i=1.833, 98 is 91/49i=1.85, 128 is 120/64i=1.875. These average out to approximately 1.82. This ratio of 1.82 units out for 1 unit up produces an angle of about 29 degrees. The value of the distance from where the crossing line occurs back to the zero x and y starting point can be easily calculated. Conjugate 50 is 50−5=45 horizontal units distance along the x axis and 25i=25 vertical units up the y axis. The diagonal recording line then equals: 45²=2,025+25²=625=2,650—the square root of which is 51.5, the distance along the average recording line. To check the slope of this value take 25/51.5=0.4854 which is close to the sine of 29 degrees. The other 63 numbers can be easily worked out. As calculated before using known data, the outer chain of the DNA molecule rises at a 29 degree slope. However, the DNA molecule and the formation of the code are separated by at least two transitions, first the messenger RNA, and second, the transcription RNA, before the code is given to the protein molecules. What this paper is saying is that the message and the slope taken from the DNA molecule relate to the mathematics used to form the amino acid selection of the code.

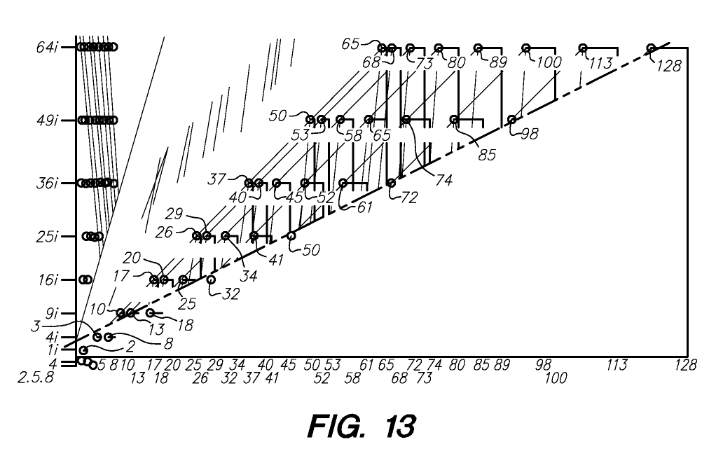

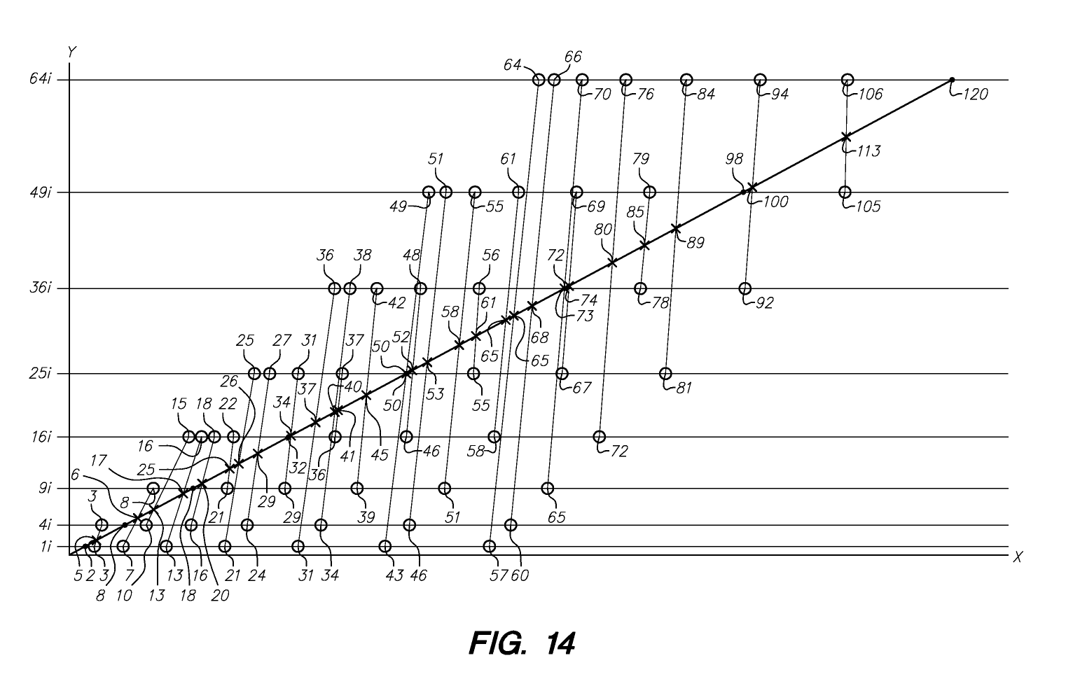

Having plotted the 8 points on the sloping line, the other 56 combinations of complex numbers shown in columns II and III, Table 15, and adapted as shown on FIG. 11, are plotted in FIG. 12. FIG. 12 also has the shorter comparative lines drawn and extended downward, thus some of the meeting points are shown. The others are shown on FIGS. 12a and 12b extending way down to the bottom the diagram. FIG. 13 is the same as FIG. 12, but is showing only the upper values of the complex conjugates with their conjugate values labeled. FIG. 12 had taken the conjugate values and labeled them along the x axis. In FIG. 13, these values are labeled on the plotted spots they are associated with, and their plotting lines as seen on FIG. 12, are only partially drawn so as not make the drawing confusing. In FIG. 13, only the conjugate 34, tied to the plotted point −3+25i has its plotting line completely shown as an example. FIG. 14 is a diagram showing the actual real position on the Argand diagram of the plotted points. Remember, on FIG. 13, the top set of conjugates are 65, 68, 73, 80, 89, 100, 113, and 128 and had the series of stubby horizontal lines connecting these conjugate numbers to what would be their vertical plotting lines. These stubby lines grow from 1 unit to 8 units in length as one moves from the left to the right, giving us an indication of how the plotted points relate to their vertical plotting lines.

In FIG. 14, derived from the same set of conjugates on the top line but shown with tiny circles and labeled with their real number positions, are 64, 66, 70, 76, 84, 94, 106, and 120. These numbers are of course, 1, 2, 3, 4, 5, 6, 7, and 8 less than the same set of conjugate numbers on that row in FIG. 13. These numbers are the actual plotted position values of the conjugates and their comparative connecting lines cross the slopping line at specific points. These points are marked with an x; the actual non-whole integer values are not shown. What is shown is the conjugate value to which they are tied. Going back to our first example, conjugate 34 has a crossing line going from the real position 31 on the 25i line down to the real position 29 on the 9i line. This line, had it been extended as explained earlier, would be used to determine its comparative point. The actual crossing points formed from the crossing lines also has, as mentioned, a real position on the Argand diagram. This real position can be calculated using the size of the sides of the triangle formed between the sloping line and the base line with a hypotenuse length from zero up to the plotted point. The importance of these values, when calculated, is that all 64 conjugate numbers with the crossing lines which also extend to become the comparative lines, are now transferred to the sloping line, in a geometrically scaled manner. A single sloping line shown in the Argand diagram in FIG. 14, has the 64 possibilities pared down to 20 possibilities. The DNA molecule with its 64 hydrogen bridge choices can tie to take this complex number single line transitioning through the RNA transcription and RNA messenger molecules. Because the information is on a single line, the varying helix twisting angles of these molecules or their varying rising slopes cannot undo this Argand plane code.

The actual position of the comparative points can be calculated where “a” is the real value component and “b” i is the imaginary component for the complex number “a” x+“b” i y with: −½x[(“a”²+“b”²)+(“a”²+“b”²)(2−“a”−“b”)+2 “a”x“b”]

For the conjugate 113−½x[(49+64)+(49+64)(2−7−8)+2x7x8]=−1470/2=−735i; “a”=7, “b”=8.

All the comparative points are negative values of i because they are below the zero y axis line. The single point conjugates using this formula have values even though they don't have converging intersecting comparative lines. All the comparative point values are shown in Table 15, column VII. The genetic code has 64 letter combinations that have a degenerative quality so they combine to represent 20 amino acids. The genetic letter combinations show there are 8 groups of 4 letters, 1 group of 3 letters, 12 groups of 2 letters and 5 groups of 1 letter for 26 different groups. However, 3 groups of 1 letter are terminating or are nonsensical, thus there are 3 fewer. In addition, when the 2 and 4 groups of Serine, Leuine, and Argine are combined thus, again there are 3 fewer groups bringing the total to the 20 different amino acids. The conjugate numbers to get down to 26 can be grouped in a degenerate manner shown in Table 15. The 2 and 1 letter combinations that are not further combined are shown in column IV, in line with their conjugate numbers. The 3 and 4 letter combinations are shown in column VI, using the 2 and 1 letter combinations of column V to further recombine, also in line with their conjugate numbers.

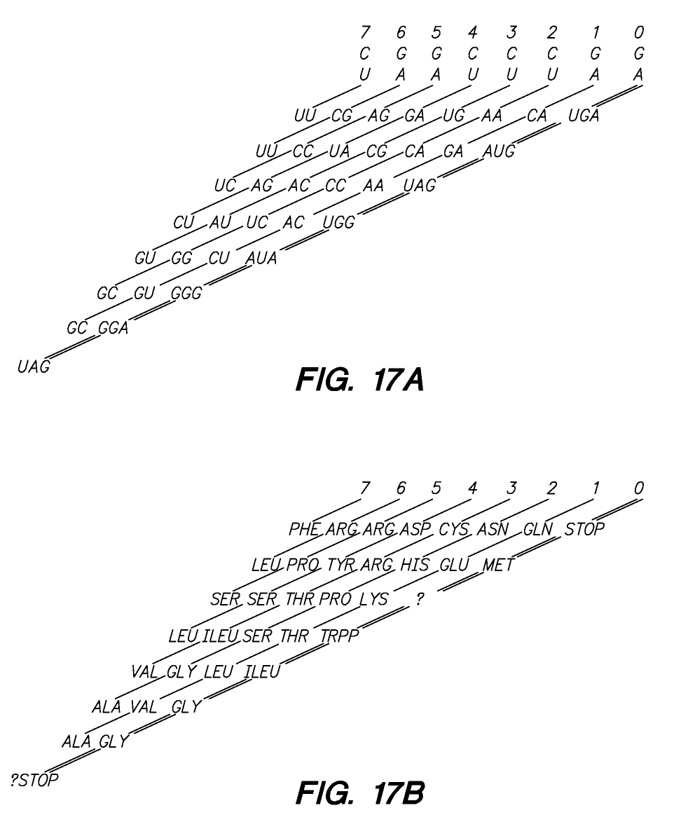

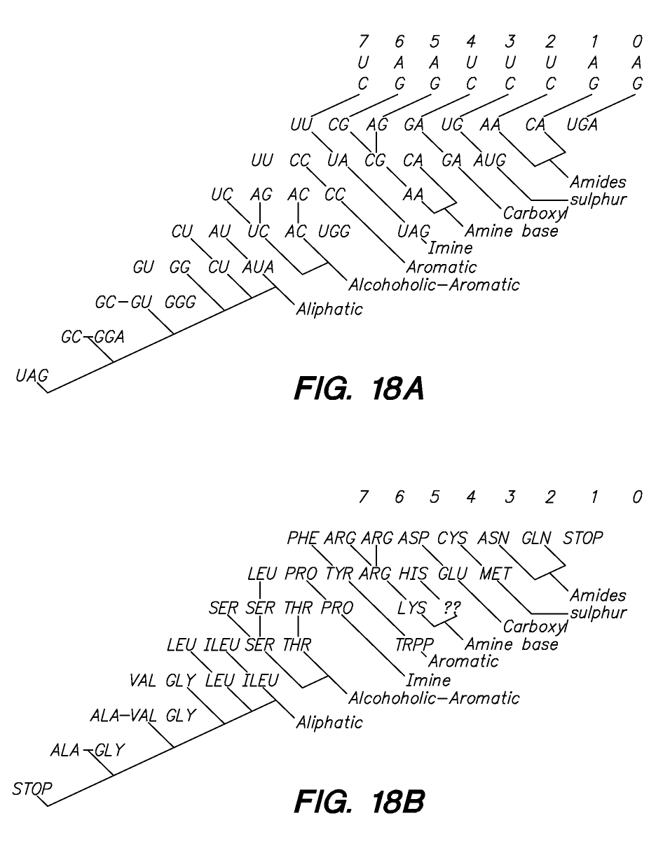

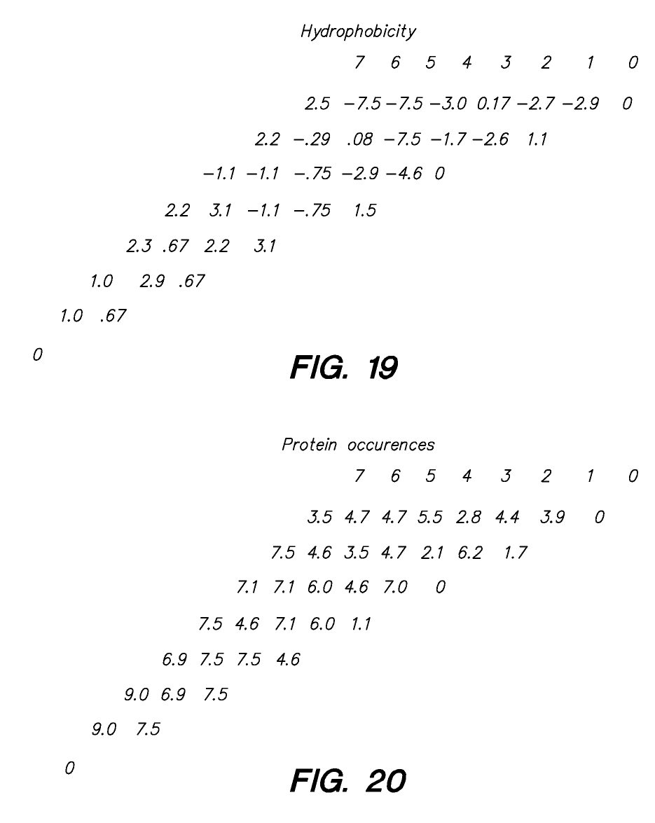

In the column line also on the far right is the list of amino acids which tie to their respective conjugate numbers. The first is a stop function which is tied to conjugate 2; then the amino acid ALA which is shown tied to conjugate 5. Then GLY to 8 combined with 18, using the combinations coming from column V regrouped together for the final combination as noted in column VI. Just after this column is the function grouping column showing how the amino acids are tied together based on their chemical properties. The first group, Aliphatic type amino acids, consists of Glysine, Alanine, Valine, Leucine, and Isoleucine. Then comes the Alcoholic, Aliphatic, and Aromatic group etc. Grouping the amino acids thus and tying them with their functions into the conjugate numbers is a continuing step in the process. The known hydrophobicity and Protein occurrence rates are shown on FIGS. 19 and 20. Later we will see the importance of the formats of FIGS. 17 through 18b which is also used on FIGS. 19 and 20 showing how the Hydrophobicity and Protein occurrence rates tend also to be organized in the same manner as those two figures.

When the complete tie between the amino acids and the conjugate numbers is done, it is vital to understand the relationship between the amino acids and the complex numbers. One amino acid-conjugate number is picked at a time for use in the Protein molecule based on information coming from the RNA molecule. The choice as directed by the RNA molecule of which amino acid-conjugate number is to be put in the Protein molecule is not effected, in any way, by the order of sequencing order on Table 15. What choice occurs before or after a selection from Table 15 is also completely up to the agenda of the RNA molecule for a Protein molecule.

Table 15 shows the match up of amino acids and conjugate numbers but not letter combinations and conjugate numbers. The genetic code is normally presented in a standard format with the codon first letter choice grouped in the left margin; the second position grouping is along the top and the not so important third position is individually listed on the back right margin. The matching of letters to amino acids in Tables 16a, 16b, and 16c uses a different format, but again the not-so-important third position has the letters individually listed on the right margins. On Table 16a, because, as is known, the last letters pair up, A-G, and C-U, there are two margins, 1 and 2 on the right side. Starting with the first letter combination, PHE would be UU with the 1 margin U brought over for UUU, or the paired letter code would be UU with the 2 margin C brought over for UUC.

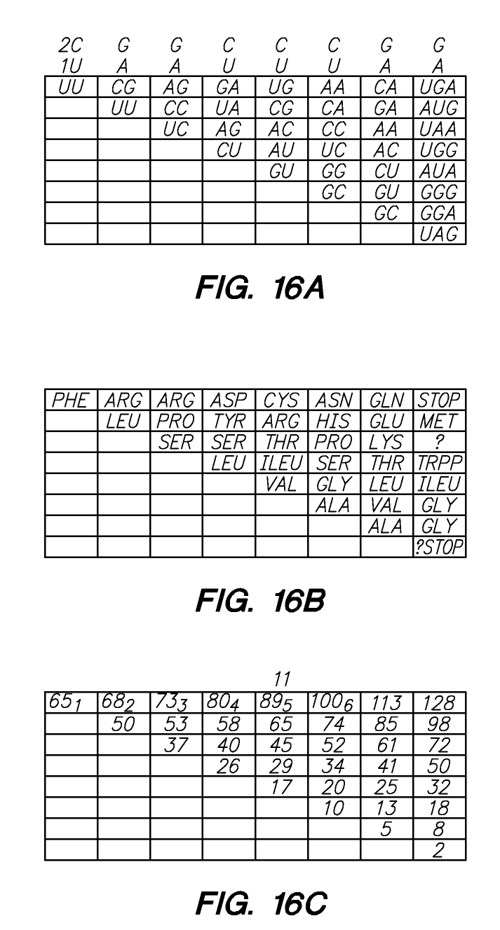

Notice that the critical aspect of this format is that the code letter selection is organized about the last letter selection. Table 16b shows the matching amino acids that work with Table 16a, and Table 16c shows the conjugate numbers that go with the amino acids. The bottom line of Table 16c shows the 8 single digit complex conjugates 2, 8, 18, 32, 50, 72, 98, and 128. Look back at FIG. 13 where this same line of numbers fall on the sloping line. On that same figure, notice that conjugate 65 is the farthest away from the sloping line numbers and that this is true on Table 16c. With this in mind, imagine taking the conjugate numbers of Table 16c, and the accompanying amino acids and letters of Tables 16b and 16a, and squashing them to match the format of FIG. 13. Tables 16b and 16a are now converted into FIGS. 17b and 17a. Remember that these figures, as did FIG. 13, represent the upper group of amino acids and letter combinations. But, also notice that the dual last letter margins of FIG. 16a are also carried over to FIG. 17a. So again, bringing down the U from margin 1, the first letter combination would again be UUU the farthest position away from the sloping line, now shown as a double line.

Imagine the whole set of dual letters on FIG. 17 as flipped over and down-positioned somewhat symmetrically below the double sloping line so as to match the corresponding positions of FIG. 12. The first letter combinations for this lower group would again be UU, but this time bring down the C from margin 2, for the letter code of UUC which is at the position farthest away from the double line. FIGS. 17a and 17b, and 18a and 18b coming up are diagrams. FIGS. 11, 12, 13, and 14 are plotted drawings on an Argand plane. FIG. 17a is again showing the last letter selection organized parallel to the sloping lines of FIGS. 13 and 14. Look back at Table 15 showing the amino acids bunched together in their chemical group functioning categories. These groupings are shown again in FIG. 18b, falling generally perpendicular to the sloping line. Then in FIG. 18a, the amino acids are replaced with their appropriate letter combinations. Again, this is critically important, as the group functions are joined perpendicularly to the sloping line, and the last letter selections are joined parallel to this line. This is a very useful format to use because it correctly suggests a fairly complete independence of the last letter selection to the group functions of the amino acids. The Hydrophobicity and especially the Protein occurrence rates of amino acids in FIGS. 19 and 20 show an organization parallel to the sloping line. Even if one were to discard the complex conjugate tie, this format used in FIGS. 12 through 20 for the genetic code has more to offer than the standard format. Also the recording line distances from the starting point on the Argand diagram are, as mentioned before, easily calculated. From the 64 choices, one of which could be selection by codon and if used along with the Fibonacci-Lucas division-position selection, would form a complete description of the DNA sequencing which could be put in a computer and tied to other information. It should be recalled that FIGS. 2 and 4 convert the Fibonacci-Lucas series into a sloping strand, while FIG. 14 converts the conjugate numbers into a different sloping strand. Using numbers from the earlier examples, the three combined concepts in terms of numbers would be 51.5-32-3×7, the first being the codon letters UGG, amino acid TRPP, the second number would place the letter group at row 32 from the starting point and the third two numbers 3×7 would mean it was the third copy from the starting position. All three sets of numbers can also represent different triangles on the Theodorus spiral, 3, 7, and 32 being the whole integers and the square root of 2,650, being a non whole integer.

What is claimed is:

1. A computer-implemented DNA sequence tracking method comprising:
identifying a DNA molecule step position taken from a starting point of a DNA sequence; and
using a Fibonacci-Lucas series, combining said DNA molecule step position with a number of times that a DNA molecule has divided, based on the starting point of the DNA sequence.

2. A method as claimed in claim 1, wherein the DNA molecule has two strands, a Fibonacci portion of the Fibonacci-Lucas series representing one strand and a Lucas portion of the Fibonacci-Lucas series representing the other strand.

3. A method as claimed in claim 2, wherein said combining comprises matching numbers in the Fibonacci portion of the Fibonacci-Lucas series with numbers in the Lucas portion of the Fibonacci-Lucas series, beginning with matching a first number in the Fibonacci portion with a first number in the Lucas portion, matching respective second numbers in the respective portions, said matching corresponding to tying of each DNA ladder step on one side to its opposite chain step on the other side.

4. A method as claimed in claim 1, wherein disassembly of respective Fibonacci numbers and Lucas numbers from the Fibonacci-Lucas series corresponds to disassembly of a DNA molecular strand.

5. A method as claimed in claim 1, when the Fibonacci-Lucas series comes apart, the number of times the molecule has divided and the location from the starting point, can be written in a number of different ways—most obvious is the row number followed by the copy number, or by using just the Fibonacci-Lucas numbers, or a combination of the two ways, such as from FIG. 3's bottom row, series number 3×7 on the 32 row.

6. A method as claimed in claim 1, further comprising representing the Fibonacci-Lucas series as a spiral corresponding to a helix of the DNA molecule, and multiplying respective quarter-circle arc segments to be circles corresponding to flat hydrogen bridge bases in said DNA molecule.

7. A method as claimed in claim 6, further comprising joining constantly changing bases of the DNA molecule to side strands, using center lines with varying joint angles whose average is π/4, corresponding to an average uniform construction of the DNA molecule at the strands.

8. A method as claimed in claim 7, further comprising tying a hydrogen bridge's weak bond electron's movement to a cycloid path proportional to 4/π, said proportion corresponding to an inverse of an average base-strand joining condition of the hydrogen bridge which varies as a function of a selected base, said relationship corresponding to successive spiral circumferences of said Fibonacci-Lucas series.

9. A method as claimed in claim 8, further comprising projecting an average cycloid path onto a circumference of two circles, said circles corresponding in turn to a projection of spirals of DNA helix strands onto a flat surface so as to form a circle.

10. A method as claimed in claim 9, wherein, in the Fibonacci-Lucas series, each series number relates to an immediately preceding and an immediately following number by an amount which converges to the “Golden Number” or 0.6180339887 . . . , wherein the Golden Number represents an actual length of a sloping DNA strand centerline projected onto a flat horizontal surface formed by a circumference line of the DNA helix.

11. A computer-implemented DNA sequence tracking method comprising:
identifying a DNA molecule step position geometrically, beginning with a starting point of a DNA sequence; and
using a Theodorus spiral with its whole number roots, combining said DNA molecule step position with a number of times that a DNA molecule has divided, based on the starting point of the DNA sequence.

12. A method as claimed in claim 11, wherein the Theodorus spiral comprises a secondary series of spirals which ties groups of three whole numbers together a manner corresponding to triplets in the DNA molecule mapping to codons used in genetic code.

13. A computer-implemented DNA sequence tracking method comprising:
converting a triplet letter formation taken from linear dimensional strands of a DNA molecule, using RNA molecules, into a multidimensional genetic code, said code being derived from a multidimensional Argand diagram and placed back on a diagonal recording line with an average slope of 29 degrees, corresponding to a slope of a strand line of the DNA molecule.

14. A method as claimed in claim 13, wherein the Argand diagram comprises 64 complex conjugate possibilities, exactly one of said possibilities being selected and tied to an amino acid, and tied also to a choice of codon letters corresponding to the triplet letter formation.

15. A method as claimed in claim 14, further comprising reducing the 64 complex conjugate number possibilities to 20 different possibilities in a manner corresponding to a reduction of 64 possible letter combinations for an amino acid combination to 26 possibilities consisting of eight 4-letter groups, one 3-letter group, twelve 2-letter groups, and five 1-letter groups, and a further reduction to 20 amino-letter triplets.

16. A method as claimed in claim 14, comprising mapping the 64 complex conjugate number possibilities and corresponding amino acid-codon triplet matches to the Argand diagram, so that the mapping is based on the first two letters of the triplets.

17. A method as claimed in claim 14, further comprising ordering the 64 complex conjugate number possibilities in a manner corresponding to an amino acid grouping.

18. A method as claimed in claim 14, further comprising grouping the 64 complex conjugate number possibilities in an order sequence related to the Argand diagram and further matching the possibilities to the amino acids according to hydrophobicity characteristics of the amino acids.

19. A method as claimed in claim 14, further comprising grouping the 64 complex conjugate number possibilities in an order sequence related to the Argand diagram and further matching the possibilities to the amino acids according to protein occurrence of the amino acids.

20. A method as claimed in claim 1, further comprising converting a triplet letter formation taken from linear dimensional strands of a DNA molecule, using RNA molecules, into a multidimensional genetic code, said code being derived from a multidimensional Argand diagram and placed back on a diagonal recording line with an average slope of 29 degrees, corresponding to a slope of a strand line of the DNA molecule, such that strand distances on the Argand diagram coupled with the Fibonacci Lucas division-position selection describe a DNA sequence.
